(19) United States
(12) Patent Application Publication
(10) Pub. No.: US 2022/0236563 A1
(43) Pub. Date: Jul. 28, 2022

(54) OPTICAL DEVICE, IMAGE DISPLAY, AND OPTOMETRIC APPARATUS

(71) Applicant: RICOH COMPANY, LTD., Tokyo (JP)

(72) Inventors: Shu TANAKA, Osaka (JP); Suguru SANGU, Kanagawa (JP); Tatsuya SHIMOKAWA, Hyogo (JP); Shuichi SUZUKI, Kanagawa (JP)

(21) Appl. No.: 17/613,085

(22) PCT Filed: Jun. 24, 2020
(86) PCT No.: PCT/IB2020/055942

§ 371 Date: Nov. 22, 2021

(30) Foreign Application Priority Data
Jun. 27, 2019 (JP) 2019-120427
Apr. 1, 2020 (JP) 2020-066158

(51) Int. Cl. G02B 27/01; G02B 5/30; G02B 26/08; G02B 26/10

(52) U.S. Cl. CPC G02B 27/017; G02B 5/3008; G02B 2027/0178; G02B 26/0833; G02B 26/101; G02B 5/3025

(57) ABSTRACT

An optical device according to at least one embodiment of the present disclosure includes a projector configured to project scanning light that is light in a predetermined polarized state. The projector included in the optical device includes an optical member configured to selectively reflect the light in the predetermined polarized state.

### TECHNICAL FIELD

The present disclosure relates to an optical device, an image display, and an optometric apparatus.

### BACKGROUND ART

In recent years, technologies and products relating to virtual reality (VR) and augmented reality (AR) are getting attention. In particular, application of AR technology to industrial fields is expected as a measure to display digital information which is an additional value in a real space. A head mounted display (HMD) available in a behavioral (working) environment is developed.

A mainstream HMD is a transmissive (see-through) HMD that causes a user to visually recognize a virtual image and a real image of an object or the like in a real space in parallel. A HMD that displays a virtual image in front of an eye via a partially reflective film or an image guide structure and a retinal rendering HMD that renders an image directly on a retina via a partially reflective film start to appear in the market.

A device that projects scanning light on the retina of an eyeball of a user via an optical part to cause the user to visually recognize an image with projected light is disclosed (for example, see PTL 1).

### CITATION LIST

### Patent Literature

[PTL 1]
JP-6209662-B

### SUMMARY OF INVENTION

### Technical Problem

The device in PTL 1, however, may not cause the user to properly visually recognize the image with the projected light and the real space.

An object of the disclosed technology is to improve visual recognizability for an image with projected light and a real space.

### Solution to Problem

An optical device according to an embodiment of the disclosed technology includes a projector configured to project scanning light that is light in a predetermined polarized state. The projector includes an optical member configured to selectively reflect the light in the predetermined polarized state.

### Advantageous Effects of Invention

With the disclosed technology, the image with the projected light can be properly visually recognized.

### BRIEF DESCRIPTION OF DRAWINGS

The accompanying drawings are intended to depict example embodiments of the present invention and should not be interpreted to limit the scope thereof. The accompanying drawings are not to be considered as drawn to scale unless explicitly noted. Also, identical or similar reference numerals designate identical or similar components throughout the several views.

FIG. 1 illustrates an example of a configuration of an image display according to a first embodiment.

FIG. 2 illustrates an example of a configuration of a scanning mirror according to the embodiment.

FIG. 3 is a block diagram illustrating an example of a hardware configuration of a controller according to the embodiment.

FIG. 4 is a block diagram illustrating an example of a functional configuration of the controller according to the embodiment.

FIGS. 5A, 5B, and 5C (FIG. 5) each illustrates an example of a configuration of a reflective liquid crystal optical element according to the embodiment.

FIG. 5B illustrates the example of the configuration of the reflective liquid crystal optical element according to the embodiment.

FIG. 6 illustrates an example of an effect of the reflective liquid crystal optical element according to the embodiment.

FIG. 7 illustrates an example of an operation of the image display according to the first embodiment.

FIG. 8 illustrates an example of a configuration of an image display according to a second embodiment.

FIG. 9 illustrates an example of an effect of an image display according to a comparative example.

FIG. 10 illustrates an example of an effect of the image display according to the second embodiment.

### DESCRIPTION OF EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise.

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

An embodiment is described below referring to the drawings. Like reference signs are applied to identical or corresponding components throughout the drawings and redundant description thereof may be omitted.

In the embodiment, scanning light in a predetermined polarized state is selectively reflected by an optical member to project an image with the scanning light. The image with the scanning light is selectively reflected with high efficiency, and hence is projected with low loss. In contrast, light from an object or the like in a real space including light other than the light in the predetermined polarized state is transmitted through the optical member with high efficiency. Thus, both a virtual image with the scanning light and a real image of the object or the like in the real space are brightly visually recognized on a surface on which scanning light is projected.

In the embodiment, an example of an image display including an optical device is described. Described here as an example of the image display is a retina projection head mounted display (HMD) that is a wearable terminal and that projects a picture or an image directly on a retina of a user with use of the Maxwellian view.

In the embodiment, an example of an image display that displays an image on the left eyeball of a user is described. However, the image display can be also applied to the right eyeball. Moreover, two image displays may be provided and applied to both eyeballs.

In the description of the embodiment, a picture is synonymous with a still picture and an image is synonymous with a movie. A laser ray is synonymous with a laser beam. A laser ray is an example of “light”.

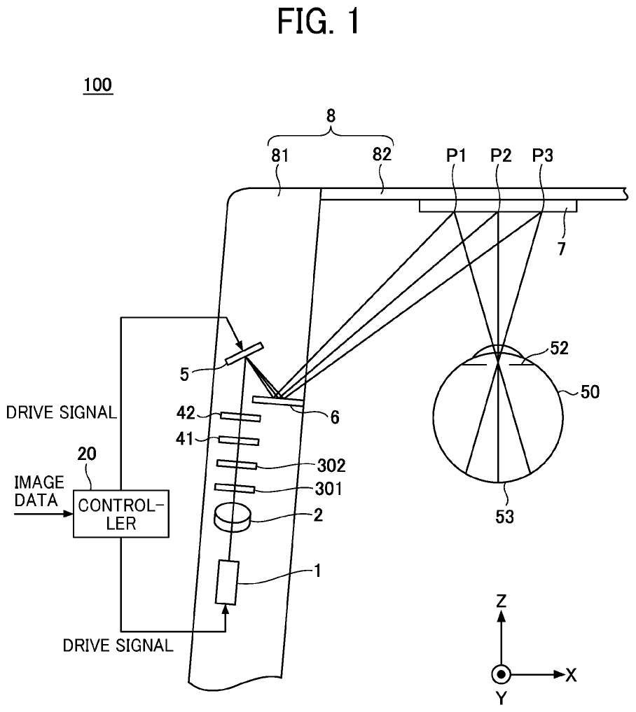

A configuration of an image display 100 according to a first embodiment is described referring to FIG. 1. FIG. 1 illustrates an example of the configuration of the image display 100.

As illustrated in FIG. 1, the image display 100 includes a laser source 1, a lens 2, an opening member 301, a light reducing element 302, a polarizer 41, a quarter wave plate 42, a scanning mirror 5, a reflecting mirror 6, and a reflective liquid crystal optical element 7. The image display 100 includes an eyeglass frame 8 and a controller 20.

The eyeglass frame 8 includes an arm 81 and a rim 82. The rim 82 holds an eyeglass lens (not illustrated). The lens 2, the opening member 301, the light reducing element 302, the polarizer 41, the quarter wave plate 42, the scanning mirror 5, and the reflecting mirror 6 are provided inside the arm 81. The reflective liquid crystal optical element 7 is provided on a surface of the eyeglass lens 8c held by the rim 82. When a user puts the eyeglass frame 8 on an ear of the user, the user can wear the image display 100 on the head.

The laser source 1 is a semiconductor laser that emits laser rays with a single wavelength or a plurality of wavelengths. The laser source 1 emits laser rays which have been time modulated in response to a drive signal from the controller 20. To render a monochrome image, a laser source that emits laser rays with a single wavelength is used. To render a color image, a laser source that emits laser rays with a plurality of wavelengths is used. In this case, the laser source 1 is an example of a “light source”.

The opening member 301 is a member having an opening that allows light to pass therethrough. The opening member 301 allows a portion of incident laser rays to pass therethrough and blocks the residual portion of the incident laser rays to shape the laser rays into a desirable sectional shape or a desirable diameter. The diameter of the opening of the opening member 301 is equal to or smaller than the diameter of laser rays collimated by the lens 2 at a light intensity of 1/e². Note that “e” is the base of natural logarithm.

The diameter of the opening member 301 is determined so that the diameter of the section of laser rays incident on the scanning mirror 5 after the laser rays pass through the opening member 301 is smaller than the effective diameter of the scanning mirror 5. In the embodiment, the opening is expected to be a circular opening; however, may be an opening partly having a distortion or having an elliptic shape. The opening member 301, for example, uniformizes the sectional light intensity distribution to bring laser rays into a desirable state, thereby improving the quality of image rays and images.

The light reducing element 302 is an optical element that reduces the light intensity of passing laser rays to obtain a proper light intensity concerning the safety of user's eye. The light reducing element 302 is, for example, a neutral density (ND) filter including a plate-shaped member made of resin, and an optical thin film provided on the plate-shaped member and having a predetermined transmissivity.

The proper light intensity concerning the safety of user's eye is, for example, a light intensity below Class 1 under the International Electro-technical Commission (IEC) 60825-1 that is an international standard relating to the safety of laser light. Since the light reducing element 302 reduces laser rays emitted from the laser source 1 to a desirable intensity, safe laser rays are projected on the retina, thereby ensuring the safety of user's eye.

The polarizer 41 is an optical element that converts the polarized state of incident light to obtain linear polarized light that oscillates in a predetermined direction. The polarizer 41 can employ a polarizing film that is sandwiched between a pair of transparent plates. The polarizing film is obtained by adding iodine into a polarizing film made of, for example, polyvinyl alcohol (PVA) and drawing the resultant to align the direction of high polymers. The pair of transparent plates can employ glass or resin such as cellulose triacetate.

The quarter wave plate 42 is an optical element that converts incident linear polarized light into one of rightward circular polarized light and leftward circular polarized light. The quarter wave plate 42 is a wave plate made of an inorganic crystal material having birefringence, such as crystal. The configuration including the polarizer 41 and the quarter wave plate 42 is an example of a “polarizing section”.

The scanning mirror 5 is a mirror that rotates around two different axes. The scanning mirror 5 rotates and changes the angle thereof to provide scanning with incident light in two different directions. In the example in FIG. 1, the scanning mirror 5 provides scanning with incident laser rays in an X direction (horizontal direction) and a Y direction (vertical direction). Since the scanning with laser rays is provided in the X and Y directions while the laser rays are synchronized, a picture or an image is projected on the retina of user's eyeball via the reflective liquid crystal optical element 7. The scanning mirror 5 is an example of a “scanner”.

Although illustration is omitted in FIG. 1, the image display 100 can include, for example, a known synchronization detection optical system to synchronize scanning with laser rays in the X and Y direction.

The X direction indicated by an arrow in FIG. 1 corresponds to a main-scanning direction in which pixels are rendered continuously in terms of time and a series of pixel groups are formed, and the Y direction corresponds to a sub-scanning direction which is orthogonal to the main-scanning direction and in which a series of pixels are arranged. The scanning speed in the main-scanning direction is higher than the scanning speed in the sub-scanning direction.

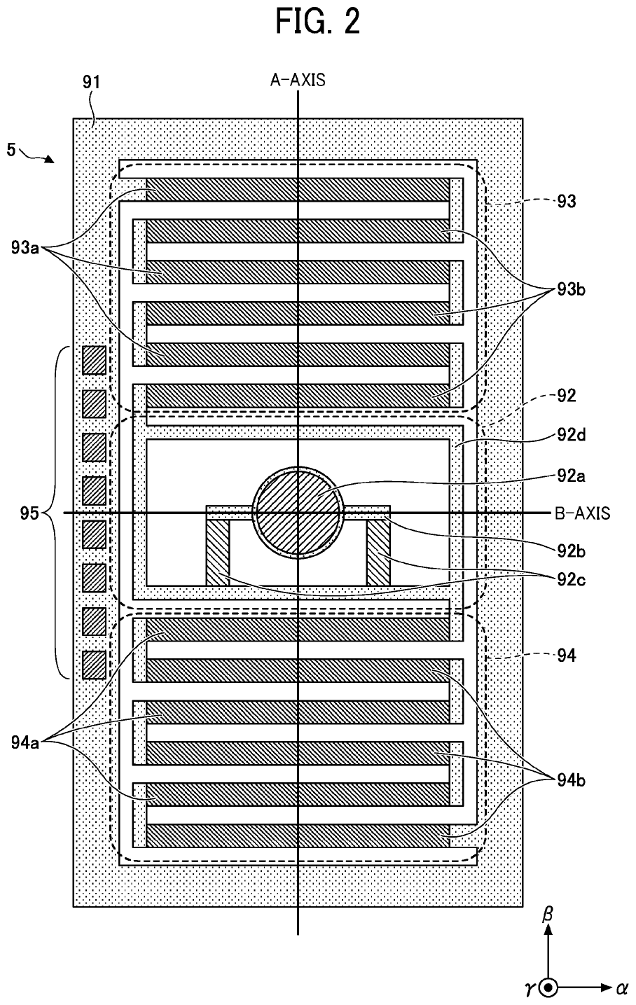

The scanning mirror 5 can use a two-axis micro electro mechanical system (MEMS) mirror. The details of the configuration of the scanning mirror 5 will be described later referring to FIG. 2.

The reflecting mirror 6 is a mirror that reflects the laser rays scanned with the scanning mirror 5 toward the reflective liquid crystal optical element 7. The surface of the reflecting mirror 6 is not limited to a flat surface, and may have a desirable shape of, for example, a concave surface or a convex surface.

The reflective liquid crystal optical element 7 is a flat-plate-shaped optical element including a liquid crystal film containing liquid crystal molecules. The reflective liquid crystal optical element 7 uses a liquid crystal molecule alignment structure including a spiral molecule array of liquid crystal molecules, a spiral pitch, and a local change in orientation to reflect (diffract) one of incident rightward circular polarized light and leftward circular polarized light and to focus the light at a position near the center of a pupil 52 of an eyeball 50.

As indicated in regions P1 to P3 in FIG. 1, the reflective liquid crystal optical element 7 reflects laser rays in different directions toward the eyeball 50 depending on the region in an X-Y plane. As described above, the reflective liquid crystal optical element 7 has a characteristic in which the magnitude of a light focusing effect on reflected light in a region differs from that in another region so that the reflected light converges at a position near the center of the pupil 52. As the magnitude of the light focusing effect increases, an effect similar to that the focal length decreases when described in terms of the function as a lens is obtained. As the magnitude of the light focusing effect decreases, an effect similar to that the focal length increases when described in terms of the lens function is obtained. In the example in FIG. 1, the magnitude of the light focusing effect increases toward the region P3 from the region P1.

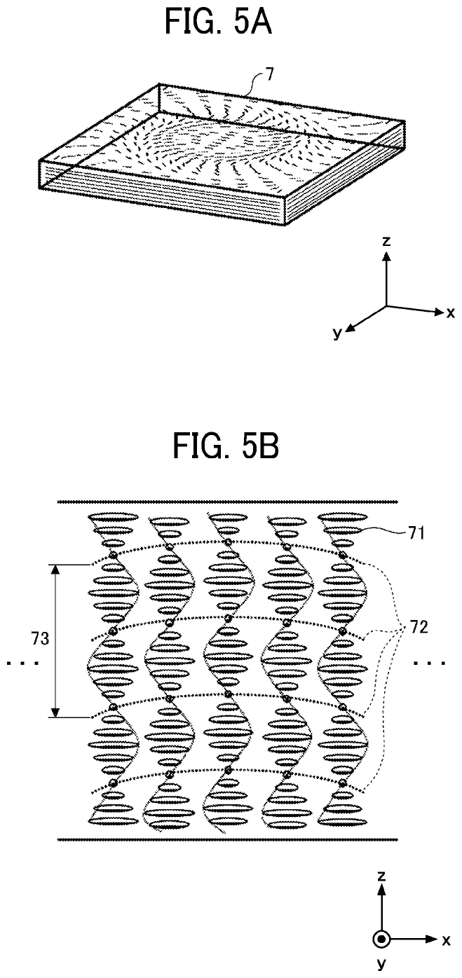

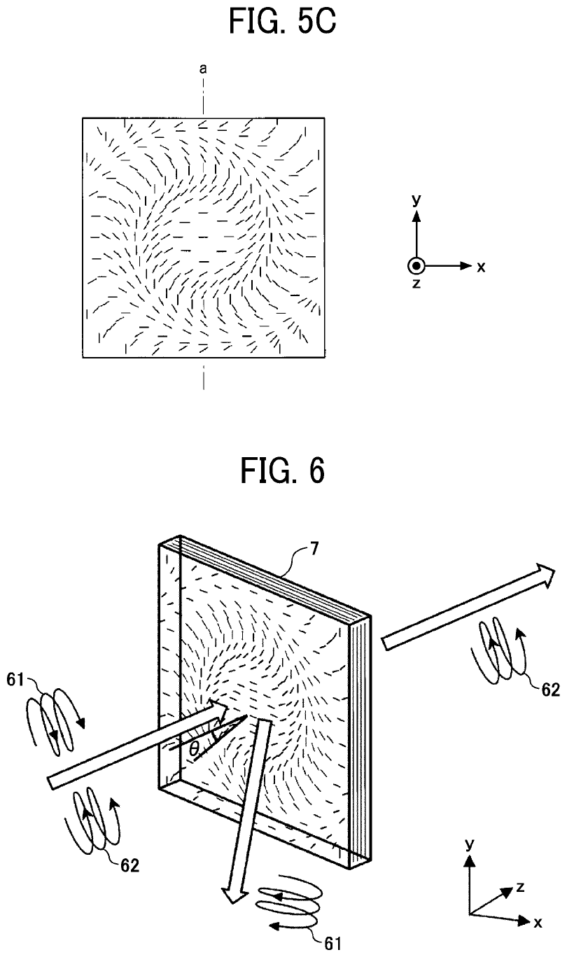

The above-described effect is derived from the liquid crystal molecule alignment structure included in the reflective liquid crystal optical element 7, and is provided by adjusting the orientation distribution of liquid crystal molecules in an element surface. The details of the configuration and effect of the reflective liquid crystal optical element 7 are described later in detail referring to FIGS. 5 to 7.

The first reflective liquid crystal optical element 7 is an example of a “first reflective liquid crystal optical element”. Moreover, the reflective liquid crystal optical element 7 is an example of an “optical member”, and further is an example of a “projector”. The element surface of the reflective liquid crystal optical element 7 is an example of a “reflecting surface”.

The controller 20 is a control device that receives an input of image data serving as a source of an image to be rendered, and that controls emission of laser light by the laser source based on the input image data. The controller 20 controls the drive of the scanning mirror 5 to control scanning with light by the scanning mirror 5.

In FIG. 1, the example has been described in which the laser source 1 and the light reducing element 302 are provided in the arm 81; however, it is not limited thereto. The laser source 1 and the light reducing element 302 may be provided outside the arm 81 to guide laser rays emitted from the laser source 1 and reduced by the light reducing element 302 to the inside of the arm 81. The controller 20 may be provided in the arm 81. Alternatively, the controller 20 may be provided outside the arm 81, and a drive signal may be supplied from the controller 20 to the inside of the arm 81.

In FIG. 1, an example has been described in which the light reducing element 302 is disposed between the opening member 301 and the scanning mirror 5; however, it is not limited thereto. The light reducing element 302 may be disposed between the opening member 301 and the lens 2, and may be disposed at a plurality of positions. The light reducing element may be omitted as far as the intensity of light to be projected on the retina of the user is safe. Proper disposition of the light reducing element 302 can downsize the image display 100.

In FIG. 1, the example has been described in which the polarizer 41 and the quarter wave plate 42 are disposed between the light reducing element 302 and the scanning mirror 5; however, the polarizer 41 and the quarter wave plate 42 may be disposed between the opening member 301 and the light reducing element 302, or between the opening member 301 and the lens 2.

In FIG. 1, the example has been described in which the reflective liquid crystal optical element 7 is provided on the surface of the eyeglass lens 8c; however, it is not limited thereto. The reflective liquid crystal optical element 7 may be provided inside or on a surface of the eyeglass lens 8c when the eyeglass lens 8c is configured as a light guide plate.

The laser source 1 is not limited to a semiconductor laser and may use a solid laser or a gas laser. The polarizer 41 may be provided with a protection film on the outermost surface of a transparent plate to improve durability or a non-reflective coating layer to prevent reflection.

When a higher optical extinction ratio is required, it is desirable to use, for example, a wire grid polarizer or a metal dispersion polarizing film.

The quarter wave plate 42 is not limited to the wave plate made of an inorganic crystal material, and may use a resin film made of an organic material, such as polycarbonate having birefringence by drawing, or a phase plate including a pair of transparent plates and a high-polymer liquid crystal phase sandwiched between the transparent plates.

The scanning mirror 5 is not limited to the MEMS mirror, and may use an optical element that can provide scanning with light, such as a polygon mirror or a galvano mirror, or a combination of these mirrors. Note that using the MEMS mirror is desirable because the image display 100 can be reduced in size and weight. The driving system of the MEMS mirror may employ any system, such as an electrostatic system, a piezoelectric system, or an electromagnetic system.

Paths of laser rays in the image display 100 are described next.

In FIG. 1, the laser rays of divergent light emitted from the laser source 1 (illustration of divergent light is omitted) is converted into substantially parallel light by the lens 2. The effect by a lens is not limited to making light substantially parallel, and may make light which has passed through a lens convergent or divergent. The substantially parallel laser rays pass through the opening member 301 and the light reducing element 302, and are converted into laser rays of rightward circular polarized light by the polarizer 41 and the quarter wave plate 42. The rightward circular polarized light is an example of a “polarized state having chirality”.

The laser rays converted into the rightward circular polarized light provide scanning in two-axis directions using the scanning mirror 5, are reflected by the reflecting mirror 6, and are incident on the reflective liquid crystal optical element 7.

For example, the reflective liquid crystal optical element 7 selectively reflects the incident laser rays of the rightward circular polarized light and causes the laser rays to be incident in the eyeball 50. The incident light in the eyeball 50 converges once at a position near the center of the pupil 52 by the light focusing function of the reflective liquid crystal optical element 7, and then forms an image on a retina 53 at a deep position of the eyeball 50. The retina 53 is an example of a “surface on which light is projected”.

The above-described visual recognition state is generally called the Maxwellian view. Light passing through a position near the center of the pupil 52 reaches the retina 53 irrespective of focus adjustment of a crystalline lens. Thus, ideally, a user can sharply visually recognize a projected image in a focused state when the user adjusts the focus of the eyes at any position in the real space. In contrast, in the actual world, laser rays incident on the eyeball 50 have a limited diameter although the diameter is small, and hence have an influence of a lens effect due to the crystalline lens. Thus, in the present embodiment of the present disclosure, design is made so that laser rays have a diameter from 350 μm to 500 μm when being incident on the eyeball 50, and an angle of divergence of beam having a positive limited value, that is, to be divergent light due to the lens 2 and the light focusing effect of the reflective liquid crystal optical element 7.

Accordingly, an image to be rendered with the laser rays through scanning using the scanning mirror 5 reaches the retina 53 via the reflective liquid crystal optical element 7 irrespective of the focus adjustment of the crystalline lens. Thus, the user can sharply visually recognize a projected image when the user adjusts the focus of the eyes at any position in the real space. In other words, the image rendered with the laser rays through scanning using the scanning mirror 5 is visually recognized in a focus-free state.

The image display 100 can change the current or voltage to be applied to the laser source 1, and can change the light intensity of laser rays to be emitted. Accordingly, the brightness of a picture or an image can be changed in accordance with the brightness of the surrounding environment in which the image display 100 is used.

The details of a configuration of the scanning mirror 5 is described next referring to FIG. 2.

FIG. 2 illustrates an example of the configuration of the scanning mirror 5. In FIG. 2, respective directions with arrows are referred to as a direction, β direction, and γ direction. As illustrated in FIG. 2, the scanning mirror 5 includes a support substrate 91, a movable portion 92, a meandering beam portion 93, a meandering beam portion 94, and an electrode coupling portion 95.

Among these portions, the meandering beam portion 93 is formed in a meandering manner to have a plurality of folding portions, and has one end coupled to the support substrate 91 and the other end coupled to the movable portion 92. The meandering beam portion 93 includes a beam portion 93a including three beams and a beam portion 93b including three beams. The beams of the beam portion 93a and the beams of the beam portion 93b are alternately formed. Each beam included in the beam portion 93a and the beam portion 93b individually includes a piezoelectric member.

Likewise, the meandering beam portion 94 is formed in a meandering manner to have a plurality of folding portions, and has one end coupled to the support substrate 91 and the other end coupled to the movable portion 92. The meandering beam portion 94 includes a beam portion 94a including three beams and a beam portion 94b including three beams. The beams of the beam portion 94a and the beams of the beam portion 94b are alternately formed. Each beam included in the beam portion 94a and the beam portion 94b individually includes a piezoelectric member. The number of beams in each of the beam portions 93a and 93b is not limited to three, and may be desirably determined.

Although the piezoelectric members included in the beam portions 93a, 93b, 94a, and 94b are not illustrated in FIG. 2, each beam may have a multilayer structure, and the piezoelectric member may be provided as a piezoelectric layer in a portion of a layer of the beam. In the following description, the piezoelectric members included in the beam portions 93a and 94a may be collectively referred to as a piezoelectric member 95a, and the piezoelectric members included in the beam portions 93b and 94b may be collectively referred to as a piezoelectric member 95b.

When voltage signals in opposite phases are applied to the piezoelectric member 95a and the piezoelectric member 95b to warp the meandering beam portion 94, adjacent beam portions are curved in different directions. The curve is accumulated, thereby generating a rotational force to rotate a reflecting mirror 92a in a reciprocating manner around an A-axis in FIG. 2.

The movable portion 92 is sandwiched between the meandering beam portion 93 and the meandering beam portion 94 in the β direction. The movable portion 92 includes the reflecting mirror 92a, a torsion bar 92b, a piezoelectric member 92c, and a support 92d.

The reflecting mirror 92a includes, for example, a base member and a metal thin film provided by vapor deposition on the base member. The metal thin film contains, for example, aluminum (Al), gold (Au), or silver (Ag). The torsion bar 92b has one end coupled to the reflecting mirror 92a, extends in the positive and negative a directions, and supports the reflecting mirror 92a rotatably.

The piezoelectric member 92c has one end coupled to the torsion bar 92b and the other end coupled to the support 92d. When a voltage is applied to the piezoelectric member 92c, the piezoelectric member 92c is deformed in a bending manner, thereby generating a twist in the torsion bar 92b. The twist of the torsion bar 92b generates a rotational force and hence the reflecting mirror 92a rotates around a B-axis.

The rotation of the reflecting mirror 92a around the A-axis causes laser rays incident on the reflecting mirror 92a to provide scanning in the α direction. The rotation of the reflecting mirror 92a around the B-axis causes laser rays incident on the reflecting mirror 92a to provide scanning in the β direction.

The support 92d surrounds the reflecting mirror 92a, the torsion bar 92b, and the piezoelectric member 92c. The support 92d is coupled to the piezoelectric member 92c and supports the piezoelectric member 92c. The support 92d indirectly supports the torsion bar 92b coupled to the piezoelectric member 92c, and the reflecting mirror 92a.

The support substrate 91 surrounds the movable portion 92, the meandering beam portion 93, and the meandering beam portion 94. The support substrate 91 is coupled to the meandering beam portion 93 and the meandering beam portion 94 to support the meandering beam portion 93 and the meandering beam portion 94. The support substrate 91 also indirectly supports the movable portion 92 coupled to the meandering beam portion 93 and the meandering beam portion 94.

The MEMS mirror constituting the scanning mirror 5 is made of silicon or glass using a micromachining technology. Using the micromachining technology can form a very small movable mirror with high precision on a substrate integrally with a driver such as the meandering beam portion.

Specifically, a silicon on insulator (SOI) substrate is shaped, for example, by etching. The reflecting mirror 92a, the meandering beam portions 93 and 94, the piezoelectric members 95a and 95b, the electrode coupling portions, and so forth are integrally formed on the shaped substrate to form the MEMS mirror. The reflecting mirror 92a and other components may be formed after the SOI substrate is shaped, or may be formed while the SOI substrate is shaped.

The SOI substrate is a substrate in which a silicon oxide layer is provided on a silicon support layer made of monocrystal silicon (Si), and a silicon active layer made of monocrystal silicon is further provided on the silicon oxide layer. The silicon active layer has a smaller thickness in the y direction than the dimensions in the α direction and the β direction. With such a configuration, a member made of the silicon active layer has a function as an elastic portion having elasticity.

The SOI substrate does not have to be planar, and may have, for example, a curvature. As long as the substrate can be integrally shaped by etching or the like and can be partly elastic, the member used for forming the MEMS mirror is not limited to the SOI substrate.

When scanning is performed in the main-scanning direction, voltages with sine waveforms in opposite phases are applied to the piezoelectric members 95a and 95b included in the scanning mirror 5, as drive signals from the controller 20. The frequency of the voltages with sine waveforms is a frequency corresponding to the resonance mode of the movable portion 92 around the A-axis. When the voltages with sine waveforms are applied, the scanning mirror 5 rotates in a reciprocating manner at a very large rotational angle with low voltage.

For the drive signals, voltage signals in a sawtooth waveform can be used. The sawtooth waveform can be generated by superposing sine waveforms. The waveform is not limited to the sawtooth waveform, and may use a waveform having rounded vertices of a sawtooth waveform or a waveform having curved linear regions of a sawtooth waveform.

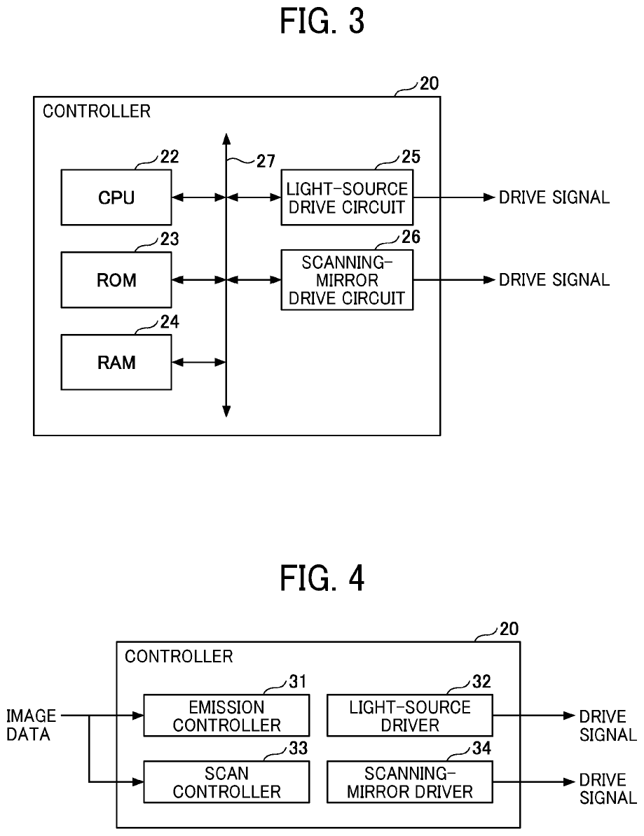

A hardware configuration of the controller 20 according to the embodiment is described next referring to FIG. 3. FIG. 3 is a block diagram illustrating an example of a hardware configuration of the controller 20.

As illustrated in FIG. 3, the controller 20 includes a central processing unit (CPU) 22, a read only memory (ROM) 23, a random access memory (RAM) 24, a light-source drive circuit 25, and a scanning-mirror drive circuit 26. These components are electrically coupled to one another via a system bus 27.

Among these components, the CPU 22 controls over the operation of the controller 20. The CPU 22 uses the RAM 24 as a work area and executes a program stored in the ROM 23 to control the entire operation of the controller 20 and implement various functions.

The light-source drive circuit 25 is an electric circuit that is electrically coupled to the laser source 1 and applies a current or a voltage to the laser source 1 to drive the laser source 1. The laser source 1 turns ON or OFF emission of laser rays or changes the light intensity of laser rays to be emitted in accordance with a drive signal that is output from the light-source drive circuit 25.

The scanning-mirror drive circuit 26 is an electric circuit that is electrically coupled to the scanning mirror 5 and applies a voltage to the scanning mirror 5 to drive the scanning mirror 5. The scanning mirror 5 changes the angle of rotation of the reflecting mirror 92a included in the movable portion 92 in accordance with a drive signal that is output from the scanning-mirror drive circuit 26.

A functional configuration of the controller 20 according to the embodiment is described next referring to FIG. 4. FIG. 4 is a block diagram illustrating an example of the functional configuration of the controller 20. As illustrated in FIG. 4, the controller 20 includes an emission controller 31, a light-source driver 32, a scan controller 33, and a scanning-mirror driver 34.

Among these components, the respective functions of the emission controller 31 and the scan controller 33 are implemented by, for example, the CPU 22. The function of the light-source driver 32 is implemented by, for example, the light-source drive circuit 25, and the function of the scanning-mirror driver 34 is implemented by, for example, the light-source drive circuit 25.

Among these components, the emission controller 31 receives an input of image data which is a base of an image to be rendered, and outputs a control signal for controlling the drive of the laser source 1 to the light-source driver 32 based on the received image data.

The scan controller 33 receives an input of image data which is a base of an image to be rendered, and outputs a control signal for controlling the drive of the scanning mirror 5 to the scanning-mirror driver 34 based on the received image data.

When an image to be visually recognized at a desirable position has a distortion or the like, the emission controller 31 and the scan controller 33 may perform control to correct a distortion or the like.

The light-source driver 32 applies a current or a voltage to the laser source 1 to drive the laser source 1 based on a control signal that is input from the emission controller 31. The scanning-mirror driver 34 applies a voltage to the scanning mirror 5 to drive the scanning mirror 5 based on a control signal that is input from the scan controller 33.

The details of the configuration of the reflective liquid crystal optical element 7 are described next referring to FIGS. 5A and 5B. FIGS. 5A and 5B illustrate an example of the configuration of the reflective liquid crystal optical element 7. FIG. 5A is a perspective view of the reflective liquid crystal optical element 7. FIG. 5B illustrates a portion of a section spatial distribution of liquid crystal directors 71 included in the reflective liquid crystal optical element 7. FIG. 5C illustrates a portion of an in-plane spatial distribution, in an element surface, of the liquid crystal directors 71 included in the reflective liquid crystal optical element 7.

As illustrated in FIG. 5, the element surface of the reflective liquid crystal optical element 7 represents an x-y plane that is a plane parallel to the liquid crystal directors 71 or the substrate surface, and the section represents a plane perpendicular to the element surface, for example, an x-z plane.

As illustrated in FIG. 5A, the reflective liquid crystal optical element 7 is formed of a flat-plate-shaped liquid crystal film. The reflective liquid crystal optical element 7 is fabricated such that a desirable molecular alignment structure is formed using a photopolymerizable liquid crystal material, then the molecule alignment structure is fixed by irradiation with UV rays, and the substrate is eliminated. Polymerization hardens the orientation and position of liquid crystal molecules while the state before polymerization is kept. Thus, the liquid crystal molecule alignment structure may represent the state before or after polymerization.

As illustrated in FIGS. 5B and 5C, the liquid crystal molecule alignment structure in which the liquid crystal directors 71 have three-dimensional periodicity is enclosed in the reflective liquid crystal optical element 7. The liquid crystal directors 71 have an average molecule alignment direction in which liquid crystal molecules are arranged with long-axis directions thereof aligned.

The liquid crystal material according to the embodiment of the present disclosure is cholesteric liquid crystal in which a chiral agent is added to nematic liquid crystal made of achiral molecules, or cholesteric liquid crystal in which liquid molecules have chirality. In cholesteric liquid crystal, a twist is induced in molecule orientation between adjacent molecules, thereby forming a spiral periodic structure having chirality in a direction perpendicular to the liquid crystal directors 71. That is, the liquid crystal directors 71 formed of liquid crystal molecules enclosed in the reflective liquid crystal optical element 7 according to the embodiment of the present disclosure form a spiral molecule array having chirality in a depth direction perpendicular to the element surface, that is, in a z direction. Cholesteric liquid crystal depends on the chirality of the spiral and hence has characteristics of Bragg reflection to selectively reflect synchronous chiral circular polarized light.

In the reflective liquid crystal optical element 7, the start position of the spiral structure, that is, the alignment direction of the liquid crystal directors 71 in the element surface is adjusted. That is, as illustrated in FIG. 5C, the in-plane orientation distribution of the liquid crystal directors 71 in the element surface of the reflective liquid crystal optical element 7 has a periodic array in which molecule orientation periodically radially changes in the element surface from a substantially center portion of the element surface. More specifically, the liquid crystal directors 71 have an orientation distribution in which the alignment direction is periodically rotated in a radial direction that can be a desirable direction from the element center portion, and the period gradually decreases from the center portion toward an edge portion, that is, the period nonlinearly changes.

Note that FIG. 5C schematically illustrates a portion of the in-plane spatial distribution, and it is not limited thereto. The in-plane spatial distribution may have a proper number of periods based on the element size and the required function.

With such an in-plane orientation distribution, for example, as illustrated in FIG. 5B, a phase distribution may be formed in the reflective liquid crystal optical element 7. In the phase distribution, an equiphase surface 72 is curved in a concave shape in the positive z direction that is the incident direction of light, in the spiral molecule array. That is, the molecular orientation distribution that locally varies provides a concave phase deviation in reflected light. Thus, the reflective liquid crystal optical element 7 has reflecting and focusing effects for light incident in the positive z direction.

As illustrated in FIG. 1, the reflective liquid crystal optical element 7 reflects laser rays in different directions toward the eyeball depending on the region in the x-y plane. When the reflective liquid crystal optical element 7 is divided along an a-axis that is parallel to the x-y plane, into a first region (x− region with respect to the a-axis) and a second region (x+ region with respect to the a-axis), the in-plane orientation distribution in the first region is asymmetric to that in the second region. More specifically, the period in the second region including the P3 region illustrated in FIG. 1 may be entirely smaller than the period in the first region including the P1 region illustrated in FIG. 1. That is, the curvature radius of the concave phase deviation which is provided over region is smaller in the second region. In other words, the magnitude of the light focusing effect is larger in the second region. As described above, the reflective liquid crystal optical element 7 includes at least two regions with different magnitudes of the light focusing effects in the element surface. Thus, the reflective liquid crystal optical element 7 can reflect incident laser rays so that the laser rays converge at a position near the center of the pupil 52. That is, the reflective liquid crystal optical element 7 functions as an aspherical surface mirror, or further a free-form surface mirror, and can provide the Maxwellian view.

When the number (the number of periods) of spiral pitches 73 illustrated in FIG. 5B is six or more, for example, it is desirable because reflection with a high efficiency of a peak reflection intensity of 90% or more can be provided.

A known technology can be applied to the technology to exhibit an optical function using a phase distribution formed of a liquid crystal molecule alignment structure like one described above (for example, Nature Photonics Vol. 10 (2016), p. 389 etc.), and hence the more detailed description is omitted here.

The phase distribution in the reflective liquid crystal optical element 7 can be adjusted by adjusting the initial alignment direction of the liquid crystal directors 71 in the element surface. Such adjustment can use a photo alignment technique. The photo alignment technique spatially divides an alignment film applied on a substrate and exposes each of the divided regions with linear polarized light polarized in a predetermined direction to spatially adjust the initial alignment direction of liquid crystal molecules.

The liquid crystal material may use one of a polymerizable liquid crystal material and a non-polymerizable liquid crystal material. The chiral agent may use one of a polymerizable chiral agent and a non-polymerizable chiral agent. One kind of a chiral agent may be used or two or more kinds of chiral agents may be combined and used. When liquid crystal molecules have chirality, the chiral agent may be omitted.

For a method of fabricating the reflective liquid crystal optical element 7 according to the embodiment of the present disclosure, a desirable molecule alignment structure is formed by using a photopolymerizable liquid crystal material, then the structure is fixed by irradiation with UV rays, and the substrate is eliminated. However, it is not limited thereto. The embodiment may be desirably changed in response to a request, such as an embodiment stacked on a transparent support substrate, or an embodiment sandwiched between transparent support substrates. In an embodiment in which a liquid crystal film is exposed to the air, a protection film or the like for increasing durability may be provided on the outermost surface. The shape of the reflective liquid crystal optical element 7 is not limited to a flat-plate shape, and may be a desirable proper shape in accordance with the form of the eyeglass lens 8c, such as a curved-surface form. In this case, the liquid crystal alignment structure of the reflective liquid crystal optical element 7 is adjusted in accordance with the form of the eyeglass lens 8c, and can reflect incident laser rays so that the laser rays converge at a position near the center of the pupil 52.

An effect of the reflective liquid crystal optical element 7 is described next referring to FIG. 6. FIG. 6 illustrates an example of the effect of the reflective liquid crystal optical element 7. FIG. 6 illustrates an example in which rightward circular polarized light 61 and leftward circular polarized light 62 are incident on the reflective liquid crystal optical element 7 having liquid crystal molecules having a rightward twist spiral array.

The reflective liquid crystal optical element 7, due to the spiral array having chirality as described above, reflects by Bragg reflection circular polarized light that is light with a predetermined wavelength band and that has the same chirality as that of the spiral rotation direction of liquid crystal molecules with high diffraction efficiency. In this case, a bandwidth Δλ in a predetermined wavelength band is determined by Δλ=Δnp cos θ, where Δn is a birefringence of a liquid crystal composition, p is a spiral pitch of liquid crystal, and θ is an incident angle of rays. The bandwidth Δλ is adjustable using the birefringence of the liquid crystal composition, and is from about 30 to 100 nm. This is very narrow compared with the bandwidth of visible light of from 380 to 780 nm.

As illustrated in FIG. 6, when a laser ray incident on the reflective liquid crystal optical element 7 is rightward circular polarized light 61 having the same chirality as that of the spiral rotation direction of liquid crystal molecules, incident laser light is selectively reflected with ideal efficiency.

The reflective liquid crystal optical element 7 transmits light with a wavelength band other than the predetermined wavelength band, and light with the predetermined wavelength band that is circular polarized light having chirality in a direction opposite pairing up with the spiral rotation direction of liquid crystal molecules. In FIG. 6, leftward circular polarized light 62 is transmitted through the reflective liquid crystal optical element 7.

While the phase deviation provided on reflected light is determined by the orientation distribution of the liquid crystal directors 71 in the element surface, a selective reflection characteristic of cholesteric liquid crystal is not lost by a change in molecule alignment direction. The reflective liquid crystal optical element 7 can reflect light that is light with the predetermined wavelength band and that is circular polarized light having the same chirality as that of the spiral array of liquid crystal molecules. In addition, the reflective liquid crystal optical element 7 can cause the reflected circular polarized light to converge at a position near the center of the pupil 52 because of the light focusing effect due to phase deviation that is determined by the in-plane molecule orientation distribution.

The spiral pitch of cholesteric liquid crystal changes with temperature. Thus, it is desirable to form the reflective liquid crystal optical element 7 using a liquid crystal film the structure of which is fixed so that the predetermined wavelength band does not change with temperature.

FIG. 6 illustrates the example of the reflective liquid crystal optical element 7 in which liquid crystal molecules form the rightward spiral array; however, in the present embodiment, a reflective liquid crystal optical element 7 in which liquid crystal molecules have a leftward spiral array may be used. In this case, the reflective liquid crystal optical element 7 selectively reflects and converges leftward circular polarized light having the same chirality as that of the orientation of the spiral rotation direction of liquid crystal molecules, and transmits light other than the leftward circular polarized light.

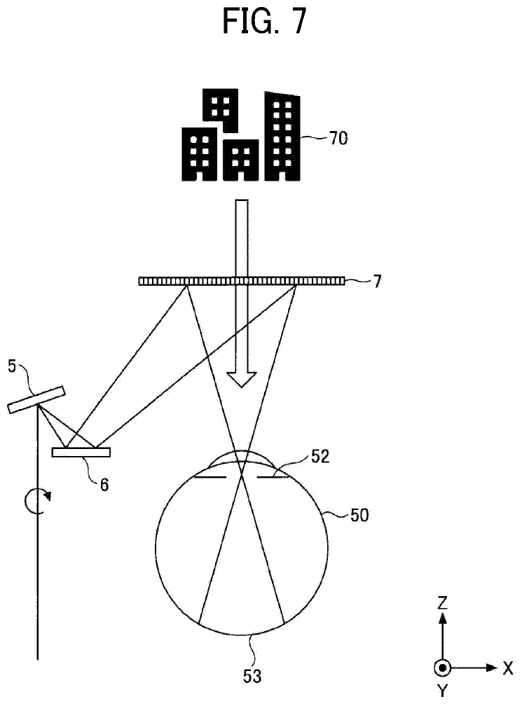

An operation of the image display 100 is described next referring to FIG. 7. FIG. 7 illustrates the operation of the image display 100.

Referring to FIG. 7, the scanning mirror 5 provides scanning with a laser ray of rightward circular polarized light, and the reflecting mirror 6 folds back the laser ray toward the reflective liquid crystal optical element 7. Then, the reflective liquid crystal optical element 7 selectively reflects the rightward circular polarized ray with ideal efficiency, converges the ray once at a position near the center of the pupil 52 of the eyeball 50 of the user, and then is projected on the retina 53 of the user. The user can visually recognize an image with the laser ray projected on the retina 53.

In contrast, light that propagates in the negative z direction from an object 70 in a real space is random polarized light with a wide wavelength band. Thus, the reflective liquid crystal optical element 7 transmits, among light from the object 70, light with a wavelength band other than the predetermined wavelength band, and transmits light of other than light having a rightward circular polarized light component, even when the light is within the predetermined wavelength band.

The bandwidth of the predetermined wavelength band at the reflective liquid crystal optical element 7 is very narrow compared with the wavelength band of visible light. Hence the reflective liquid crystal optical element 7 has good transmissivity. Thus, a major portion of light propagating from the object 70 in the real space toward the eyeball 50 is transmitted through the reflective liquid crystal optical element 7, and reaches the retina 53 of the user. Accordingly, the image of the object 70 in the real space is visually recognized with sufficient brightness.

In this way, the user wearing the image display 100 can visually recognize a virtual image and a real image of an object in a real space in parallel, and can visually recognize both the virtual image and the real image in the real space in a bright state.

Related art discloses a device that projects scanning light on a retina of an eyeball of a user via an optical part to cause the user to visually recognize an image with projected light. However, in an image display of the related art, such as a transmissive HMD that causes a virtual image and a real image of, for example, an object in a real space to be visually recognized in parallel has a trade-off relationship between brightness of a real image of the object or the like in the real space transmitted through an eyeglass and brightness of a virtual image reflected by the eyeglass. Thus, when the real image of the object or the like in the real space is brightened, the projected virtual image is darkened, and the virtual image may not be properly visually recognized.

In the present embodiment, the reflective liquid crystal optical element 7 selectively reflects scanning light of rightward circular polarized light and projects an image with the scanning light. In contrast, the reflective liquid crystal optical element 7 transmits light from a real space with high efficiency. Thus, the user with a virtual image projected on his/her retina can brightly visually recognize both the virtual image and the real image of the object or the like in the real space. In other words, visual recognizability for an image with projected light and a real space can be increased.

In the present embodiment, since an image is rendered directly on the retina of the user using the Maxwellian view, the user can be visually recognize the image in parallel and sharply when the user focuses at any position in the real space. Accordingly, for example, when the user is a worker at a manufacturing site, the user can properly visually recognize a digital content such as a work instruction in a clear field of view without an interruption of a work in a real space, and can work without visual stress because of the focus-free state.

In the present embodiment, using a flat-plate-shaped and thin reflective liquid crystal optical element 7 can reduce the image display 100 in size, and allows the image display 100 to be easily mounted.

In the present embodiment, the reflective liquid crystal optical element 7 includes the liquid crystal molecule alignment structure in which the magnitude of the focusing effect varies depending on the region. Thus, a laser ray can be properly converged at a position near the center of the pupil 52, thereby providing the Maxwellian view.

When the number of the spiral pitches 73 in the liquid crystal molecule spiral array is six or more, it is desirable because the laser ray can be reflected with further high efficiency.

In the present embodiment, the HMD is described as an example of the image display. However, the image display such as a HMD is not limited to one that is directly mounted on the head of a user, and may be one that is indirectly mounted on the head of a user via a member such as a securing portion.

In the present embodiment, the example of using the reflective liquid crystal optical element 7 in which liquid crystal molecules form the rightward spiral array is described; however, a reflective liquid crystal optical element 7 in which liquid crystal molecules form a leftward spiral array may be used. In this case, a laser ray from the laser source 1 is converted into leftward circular polarized light by the polarizer 41 and the quarter wave plate 42 and is incident on the reflective liquid crystal optical element 7, thereby obtaining an advantageous effect similar to that described above.

In the present embodiment, the example of using the reflective liquid crystal optical element 7 having one layer is described; however, a plurality of reflective liquid crystal optical elements 7 stacked in a multilayer form may be used. For example, a reflective liquid crystal optical element 7 may include three layers including a reflective liquid crystal optical element having a predetermined wavelength band of red (R), a reflective liquid crystal optical element having a predetermined wavelength band of green (G), and a reflective liquid crystal optical element having a predetermined wavelength band of blue (B). Hence, a full-color image can be projected on the retina using RGB laser sources.

An image display 100a according to a second embodiment is described.

The state of a laser ray incident on the eyeball may change in the field of view due to the function of converging reflected light by the reflective liquid crystal optical element. In this case, the state of a laser ray includes the diameter of laser ray and the angle of divergence of beam. When an image is projected at a viewing angle at which vignetting due to an eyeball motion does not occur, the state of the laser ray incident on the eyeball is desirably uniformized within a range where an image is projected on the retina.

In the present embodiment, a laser ray is incident on a reflective liquid crystal optical element via a correction reflective liquid crystal optical element to uniformize the state of the laser ray that is reflected by the reflective liquid crystal optical element and is incident on the eyeball. A configuration of the image display 100a according to the second embodiment is described.

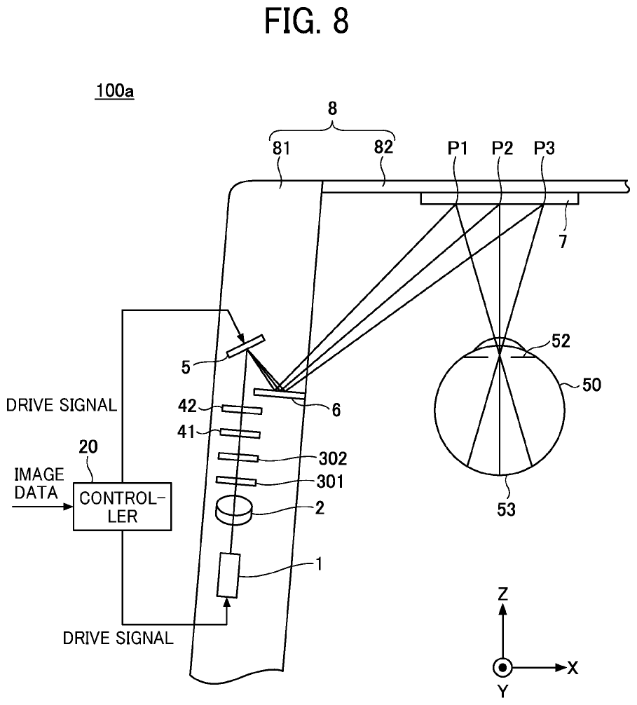

FIG. 8 illustrates an example of the configuration of the image display 100a. The image display 100a includes a correction reflective liquid crystal optical element 9. The correction reflective liquid crystal optical element 9 is an example of a “second reflective liquid crystal optical element”.

Like the reflective liquid crystal optical element 7, the correction reflective liquid crystal optical element 9 is a flat-plate-shaped optical element that reflects circular polarized light having the same chirality as that of the spiral rotation direction of liquid crystal molecules with a predetermined wavelength band, with high efficiency and focuses the light. A light focusing effect determined by the in-plane orientation distribution of liquid crystal molecules included in the correction reflective liquid crystal optical element 9 is adjusted to uniformize the state of laser rays that are incident on the eyeball 50 within a range where an image is projected on the retina 53.

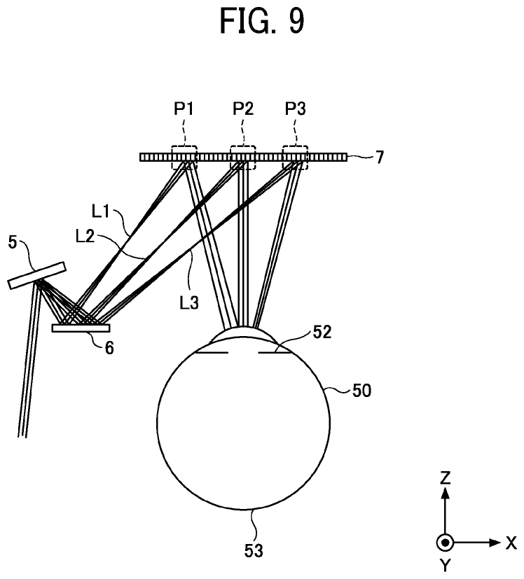

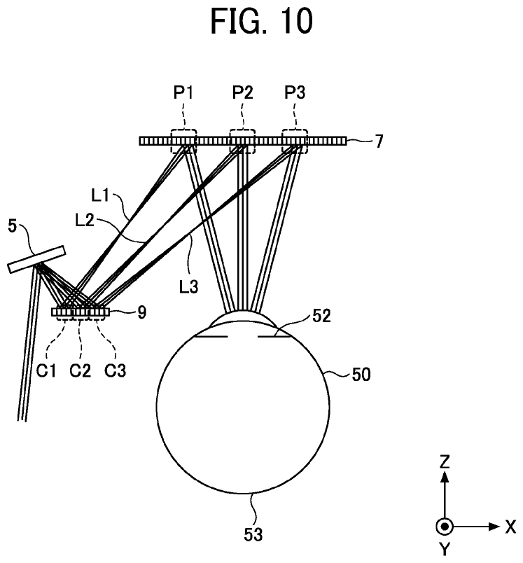

Before the effect of the correction reflective liquid crystal optical element 9 is described, an image display according to a comparative example is described referring to FIG. 9. FIG. 9 illustrates an example of an effect of an image display according to a comparative example.

Referring to FIG. 9, a scanning mirror 5 provides scanning with laser rays L1 to L3 that are reflected by a reflecting mirror 6 and then are incident on a reflective liquid crystal optical element 7. In this case, the laser ray L2 is a laser ray corresponding to the center of an image. The laser ray L1 is a laser ray corresponding to one end of the image in the X direction, and the laser ray L3 is a laser ray corresponding to the other end of the image in the X direction. In other words, the laser ray L1 corresponds to one end of a range of a retina 53 where the image is projected, and the laser ray L3 corresponds the other one end of the range of the retina 53 where the image is projected.

The laser ray L1 is reflected in a region P1 of the reflective liquid crystal optical element 7 and is incident on an eyeball 50. The laser ray L2 is reflected in a region P2 of the reflective liquid crystal optical element 7 and is incident on the eyeball 50. The laser ray L3 is reflected in a region P3 of the reflective liquid crystal optical element 7 and is incident on the eyeball 50.

As described above, in the reflective liquid crystal optical element 7, to reflect the laser rays toward the eyeball 50, converge the laser rays at a position near the center of the pupil, and then project the rays on the retina 53, the regions P1 to P3 are sequentially arranged so that the magnitude of the light focusing effect increases in the positive X direction.

As illustrated in FIG. 9, when a reflective liquid crystal optical element is arranged in front of the eyeball 50, the optical path length increases in the order of the laser rays L1 to L3. The states of laser rays when being incident on the eyeball 50 differ from one another among the laser rays L1 to L3.

For example, when it is expected that the laser ray L2 passing through the center of the field of view is incident on the eyeball 50 in a state substantially parallel to a Z-axis in FIG. 9, the laser ray L1 is incident on the eyeball in a state more divergent compared with the laser ray L2. In contrast, the laser L3 is incident on the eyeball in a state more convergent compared with the laser ray L2. In this way, with the image display according to the comparative example, the state of laser rays incident on the eyeball 50 becomes non-uniform within the range where the image is projected, and a resolution characteristic and a focus characteristic may not be uniformized.

An image display 100a according to the present embodiment is described next referring to FIG. 10. FIG. 10 illustrates an example of an effect of the image display 100a.

Referring to FIG. 10, a laser ray reflected in a region C1 of a correction reflective liquid crystal optical element 9 is incident on a region P1 of the reflective liquid crystal optical element 7. A laser ray reflected in a region C2 of the correction reflective liquid crystal optical element 9 is incident on a region P2 of the reflective liquid crystal optical element 7. A laser ray reflected in a region C3 of the correction reflective liquid crystal optical element 9 is incident on a region P3 of the reflective liquid crystal optical element 7.

The reflective liquid crystal optical element 7 and the correction reflective liquid crystal optical element 9 are made of the same liquid crystal material, and the liquid crystal molecules form a rightward spiral array having chirality the same as the chirality of polarized light in correspondence with the laser ray of the incident rightward circular polarized light. As described above, the liquid crystal molecule alignment structure is designed so that the correction reflective liquid crystal optical element 9 cancels the reflective liquid crystal optical element 7 in terms of the magnitude of the light focusing effect to uniformize the state of laser rays incident on the eyeball 50 within a range where an image is projected.

More specifically, the in-plane orientation distribution of liquid crystal molecules is determined so that the reflective liquid crystal optical element 7 has a light focusing effect having a magnitude that increases in the order of the regions P1 to P3 in the positive X direction, and the correction reflective liquid crystal optical element 9 has a light focusing effect having a magnitude that increases in the order of the regions C3 to C1 in the negative X direction.

With such a configuration, a laser ray L1 that is reflected in the region C1 having a large magnitude of the light focusing effect of the correction reflective liquid crystal optical element 9 is incident on a region P1 having a small magnitude of the light focusing effect of the reflective liquid crystal optical element 7; and a laser ray L3 that is reflected in the region C3 having a small magnitude of the light focusing effect of the correction reflective liquid crystal optical element 9 is incident on a region P3 having a large magnitude of the light focusing effect of the reflective liquid crystal optical element 7.

Accordingly, the balance between the magnitudes of the light focusing effects is adjusted in each region, and as illustrated in FIG. 10, the state of the laser ray that is reflected by the reflective liquid crystal optical element 7 and is incident on the eyeball 50 as well as the diameter of laser rays and the angle of divergence of beam are uniformized.

Also with the image display 100a according to the present embodiment, like the above-described image display 100, the incident light in the eyeball 50 converges once at a position near the center of the pupil 52 by the light focusing function of the reflective liquid crystal optical element 7, and then projects an image using the Maxwellian view which forms an image on the retina 53 at a deep position of the eyeball 50. Thus, in the present embodiment, design is made such that laser rays have, as desirable conditions for the Maxwellian view, a diameter from 350 μm to 500 μm when the laser rays are incident on the eyeball 50, and an angle of divergence of beam of a positive limited value, that is, to be divergent light due to the lens 2, and the light focusing effects of the correction reflective liquid crystal optical element 9 and the reflective liquid crystal optical element 7.

As described above, in the present embodiment, the laser rays are incident on the reflective liquid crystal optical element 7 via the correction reflective liquid crystal optical element 9. Accordingly, the state of the laser rays that are reflected by the reflective liquid crystal optical element 7 and are incident on the eyeball 50 can be uniformized to cause the user to visually recognize an image having uniform resolution characteristics and focus characteristics within the rage where the image is projected.

In the present embodiment, using the flat-plate-shaped and thin correction reflective liquid crystal optical element 9 can reduce the image display 100a in size and weight, and allows the image display 100a to be easily mounted. Advantageous effects other than the above are similar to those described in the first embodiment.

An optometric apparatus according to a third embodiment is described next.

For example, the optical device and the image display according to the embodiments of the present disclosure can be also applied to an optometric apparatus. The optometric apparatus represents an apparatus capable of performing various inspections, such as an eyesight inspection, an ocular refractive-power inspection, an ocular tension inspection, and an ocular axial length inspection. The optometric apparatus is an apparatus that can inspect an eyeball in a non-contact manner. The optometric apparatus includes a support that supports the face of a subject, an ocular inspection window, a display section that projects inspection information on the eyeball of the subject during the ocular inspection, a controller, and a measurement section. The subject secures the face at the support and stares at the inspection information projected on the display section through the ocular inspection window. At this time, the optical device according to the present embodiment can be used for the display section. Moreover, using the image display according to the present embodiment implements an optometric apparatus in a form of glasses. Accordingly, a space for inspection and a large optometric apparatus are no longer required and an inspection is available with a simple configuration in any place.

The optical device, image display, and optometric apparatus according to the embodiments have been described above; however, the present disclosure is not limited to the above-described embodiments and can be modified and improved in various ways within the scope of the present disclosure.

In the present embodiment, the HMD in the form of glasses is described as an example of the image display. However, the image display such as a HMD is not limited to one that is directly mounted on the head of a “person”, and may be one that is indirectly mounted on the head of a “person” via a member such as a securing portion.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention.

This patent application is based on and claims priority pursuant to Japanese Patent Application No. 2019-120427, filed on Jun. 27, 2019 and Japanese Patent Application No. 2020-066158, filed on Apr. 1, 2020, in the Japan Patent Office, the entire disclosure of which are hereby incorporated by reference herein.

### REFERENCE SIGNS LIST

1 laser source
2 lens
301 opening member
302 light reducing element
41 polarizer
42 quarter wave plate
5 scanning mirror (example of scanner)
6 reflecting mirror
7 reflective liquid crystal optical element (example of optical member, example of projector, example of first reflective liquid crystal optical element)
71 liquid crystal director
72 equiphase surface
8 eyeglass frame
81 arm
82 rim
9 correction reflective liquid crystal optical element (example of second reflective liquid crystal optical element)
20 controller
22 CPU
23 ROM
24 RAM
25 light-source drive circuit
26 scanning-mirror drive circuit
27 system bus
31 emission controller
32 light-source driver
33 scan controller
34 scanning-mirror driver
35 pupil position estimator
36 posture controller
37 stage driver
50 eyeball
52 pupil
53 retina
61 rightward circular polarized light
62 leftward circular polarized light
100 image display
P reflection point

1. An optical device comprising:
a projector to project scanning light that is light in a predetermined polarized state, the projector including:
an optical element to selectively reflect the light in the predetermined polarized state.

2. The optical device according to claim 1, wherein the light in the predetermined polarized state is light in a polarized state having chirality.

3. The optical device according to claim 1, wherein:
the light in the predetermined polarized state is light in a polarized state having chirality,
the light in the polarized state having the chirality is one of rightward circular polarized light and leftward circular polarized light, and
the optical element is a first reflective liquid crystal optical element.

4. The optical device according to claim 1, wherein the optical element has a surface to selectively reflect the light.

5. The optical device according to claim 1, wherein the optical element has a first surface to focus the light.

6. The optical device according to claim 5, wherein the optical element has a second surface opposite to the first surface and transmits light in a polarized state that is from the second surface and that has chirality pairing up with the chirality of the light in the predetermined polarized state.

7. The optical device according to claim 1, wherein the optical element is made of a polymerizable liquid crystal material.

8. The optical device according to claim 1, wherein:
the optical element is a first reflective liquid crystal optical element,
the first reflective liquid crystal optical element includes a liquid crystal molecule alignment structure having a three-dimensional periodicity,
the liquid crystal molecule alignment structure has a spiral molecule array having chirality in an element depth direction, and a periodic array having molecule orientation that periodically changes along and within an element surface from an element center portion in an element in-plane direction, and
the periodic array has a period that nonlinearly changes along and within the element surface from the element center portion.

9. The optical device according to claim 8, wherein:
the periodic array includes a first region and a second region that are divided with respect to the element center portion, and
the periodic array in the first region is asymmetric to the periodic array in the second region.

10. The optical device according to claim 8, wherein a number of periods of the spiral molecule array is six or more.

11. The optical device according to claim 1, further comprising:
a scanner to irradiate the projector with the scanning light, the scanner including:
a scanning mirror to rotate around two different axes; and
a reflecting mirror to reflect light reflected by the scanning mirror.

12. The optical device according to claim 11, wherein the reflecting mirror is a second reflective liquid crystal optical element having a reflecting surface to selectively reflect and focus one of rightward circular polarized light and leftward circular polarized light.

13. The optical device according to claim 1, wherein:
the optical element is a first reflective liquid crystal optical element, and
the first reflective liquid crystal optical element includes at least two regions within an element surface, the regions having different magnitudes of light focusing effects.

14. The optical device according to claim 13, wherein:
a scanner to irradiate the projector with the scanning light includes a second reflective liquid crystal optical element to selectively reflect and focus one of rightward circular polarized light and leftward circular polarized light,
the second reflective liquid crystal optical element includes at least two regions having different magnitudes of light focusing effects within an element surface, and
one of the regions having a smaller magnitude of the light focusing effect is provided closer to a surface on which light is projected by the projector compared with the other one of the regions having a larger magnitude of the light focusing effect.

15. An image display comprising:
a light source;
the optical device according to claim 1; and
a polarizing section to convert light from the light source into the light in the predetermined polarized state.

16. An optometric apparatus comprising:
at least one of the optical device according to claim 1; and
an image display which includes:
a light source, and
a polarizing section to convert light from the light source into the light in the predetermined polarized state.
